(19) United States
(12) Patent Application Publication
(10) Pub. No.: US 2008/0003565 A1
(43) Pub. Date: Jan. 3, 2008

(54) Viral nucleic acid microarray and method of use

(75) Inventors: Cassio S. Baptista, Frederick, MD (US); Xiaolin Wu, Gaithersburg, MD (US); David J. Munroe, Frederick, MD (US)

Correspondence Address: EDWARDS ANGELL PALMER & DODGE LLP;(CLIENT REFERENCE NO. 47992), PO BOX 55874, BOSTON, MA, 02205, US

(73) Assignee: Government of the US, as represented by the Secretary, Department of Health and Human Services

(21) Appl. No.: 11/800,080

(22) Filed: May 2, 2007

Related U.S. Application Data
(60) Provisional application No. 60/797,344, filed on May 2, 2006.

(51) Int. Cl. C12Q 1/70

(52) U.S. Cl. 435/5

(57) ABSTRACT

The present invention relates generally to methods of detecting and identifying known and unknown viruses using hybridization microarrays to known conserved and non-conserved viral nucleotide sequences, the sequencing of nucleotides which hybridize to the microarrays and analysis of the hybridized sequences with existing databases, thus identifying existing or new subtypes of viruses.

### REFERENCE TO RELATED APPLICATIONS

This application claims priority to U.S. Provisional Patent Application Ser. No. 60/797,344 filed on May 2, 2006, which is incorporated herein in its entirety.

### STATEMENT OF FEDERALLY SPONSORED RESEARCH

Research supported in this application was carried out by the United States of America as represented by the Secretary, Department of Health and Human Services.

### FIELD OF THE INVENTION

The present invention relates to the use of novel nucleic acid microarrays for the identification of existing and new subtypes of mammalian and avian viruses.

### BACKGROUND OF THE INVENTION

Viral screening is of great importance in terms of safety in: bio-defense, drug production, agriculture, maintaining animal stocks. Moreover, some viral infections can become serious public health issues worldwide. Therefore, detection and identification of viral pathogens related to human diseases are relevant to public health and biomedical research. Methods for detecting unknown pathogens tend to be less sensitive, requiring a relatively large amount of pathogen to be present to allow for detection.

Culturing of viruses requires an appropriate host and is frequently difficult in vitro, making detection and identification of viral pathogens more difficult than bacterial, fungal, or other pathogens that can grow independent of a host. A number of methods such as those based on the polymerase chain reaction (PCR), immunoassays, and nucleic acid microarrays have been developed for the detection of single pathogens, such as viruses. Although such assays can provide useful information regarding the presence or absence of a virus or a member of a group of related viruses, the correct test must be selected to detect the virus. This can be achieved when specific signs or symptoms are associated with infection by the virus present. However, such assays cannot be used to screen for unknown viruses, those not associated with specific diseases or conditions, or to detect contamination of surfaces or other biological samples. Moreover, multiple tests must be performed to detect multiple viruses.

Microarray methods have been developed to identify unknown viral pathogens using conserved viral sequences to allow for the detection of the largest possible number of viruses (see, e.g., Wang et al., PloS Biology, 1:257, 2003; and Wang et al., PNAS, 99:15687, 2002). Using nucleic acid sequence data, the most highly conserved sequences from each viral family of interest were used to create a microarray. This allows for the detection of members of viral families for which sequences are not specifically included in the microarray sequences as highly similar to the conserved sequences are present in at least a majority of members of each viral family or group. Further characterization must be performed to identify the specific viral type.

### SUMMARY OF THE INVENTION

The present invention relates generally to viral nucleic acid microarrays and methods of detecting and identifying known and unknown viruses using the microarrays containing known viral nucleotide sequences. The methods can further include the sequencing of nucleic acids that hybridize to the microarrays and analysis of the hybridized sequences with existing databases, thus identifying existing or new subtypes of viruses.

More specifically, the present invention relates to microarrays comprising a surface with a plurality of n-mer nucleotides capable of hybridizing to both conserved and non-conserved nucleotide regions of known viruses. In a preferred embodiment, the plurality of n-mer viral nucleotides are comprised of both conserved and non-conserved nucleotide regions of the viruses listed in Table 1.

The invention relates to microarrays comprising a surface with a plurality of n-mer nucleotides capable of hybridizing to conserved and non-conserved nucleotide regions of all known viruses as of May 2, 2006. For example, the microarrays are comprised of conserved and non-conserved nucleotide regions of the viruses listed in Table 1. Examples of n-mer oligonucleotides from the viruses in Tables 1 and 2 are provided in Table 2. Viral sequences identified after that date can also be incorporated into the microarrays of the instant invention. Conserved and non-conserved viral sequences can be selected for incorporation into a microarray based on association of the virus or family of viruses with a particular disease or disorder, such as an acquired immunodeficiency disease. Conserved and non-conserved sequences can alternatively be selected based on viral host or conditions under which the suspected virus is grown.

The invention further relates to methods for identifying known and unknown subtypes of mammalian and avian viruses using the microarrays of the invention.

More specifically, the present invention relates to a method for identifying known and unknown subtypes of mammalian and avian viruses comprising the steps of:

obtaining a microarray of the invention comprising a plurality of n-mer nucleotides on a surface for hybridizing to both conserved and non-conserved regions of known viruses;

isolating nucleic acids from a sample suspected of containing a viral nucleic acids and labeling the nucleic acids with a detectable marker;

contacting the labeled nucleic acids from the sample with the support with immobilized known conserved and non-conserved n-mer nucleotides, and incubating the support under conditions to permit hybridization of the labeled nucleic acids to the n-mer oligonucleotides attached to the support;

washing the support;

detecting labeled nucleic acids hybridized to the n-mer nucleotides;

identifying the sample nucleic acids based on their location on the support;

and optionally analyzing the sequences of the detected hybridized sample nucleic acids and comparing the sequences with a database to confirm the identity of the bound sequence or identify the virus or new subtype virus. Analyzing can include, for example, sequencing or analysis of all of the sites at which the sample nucleic acid is hybridized, or both. Importantly, the method includes the detection of viral DNA without amplification using sequence specific primers (e.g., polymerase chain reaction).

The invention further relates to methods for detection and identification of a virus in a biological sample or subject. For example the methods include diagnosing a patient with a viral infection comprising the method of obtaining an array of a plurality of n-mer viral nucleotides of conserved and non-conserved regions of all known viruses as of May 2, 2006, such as the array of the invention, immobilized on a surface; preparing nucleic acids from a sample containing or suspected of containing a virus and labeling the nucleic acids with a detectable marker; applying the labeled nucleic acids from the sample to the surface with the immobilized known conserved and non-conserved n-mer viral nucleotides, and incubating under conditions to permit hybridization of said labeled nucleic acids thereto; washing the surface, detecting hybridization of the labeled nucleic acids and identifying the virus present by the position of the bound labeled nucleic acids on the array. The methods may further comprise and analyzing the sequences of the detected hybridized nucleic acids and comparing the sequences with a database to identify the virus or new subtype virus wherein the virus is identified. The invention also includes detection and identification in biological samples such as tissue culture lines, animal colonies, viral stocks for the preparation of vaccines or other purposes.

The invention relates to methods for detection of contaminants in viral stocks and cell lines, including screening and monitoring of stocks for the presence of contaminants. The method includes isolation of nucleic acids from viral or cells stocks, labeling the nucleic acids, contacting the labeled nucleic acids with a microarray of the invention, and detecting the presence of a labeled nucleic acid hybridized to the array per the methods of the invention. Such methods can be used for the detection of aberrations in virus stocks, such as those used for the generation of vaccines. Aberrations include spontaneous mutations, point mutations or recombinations, for example with the host genome, and contaminants in viral stocks. Viral stocks can be assayed for the presence of contaminants on a regular, periodic basis, or sporadically.

The invention further relates to methods to detect genetic drift in a viral population to determine the presence or rates of mutation of one or more viruses under various conditions. The method includes isolation of nucleic acids from viruses in culture or from samples from viral hosts including samples of tissue and/or bodily fluid; labeling the nucleic acids; contacting the labeled nucleic acids with a microarray of the invention; and detecting the presence of a labeled nucleic acid hybridized to the array per the methods of the invention. For example, treatment with antiviral therapeutics can result genetic drift in the development of alterations in the viral sequence. Methods of detection of the invention can be applied to populations in the event of a large scale outbreak of infection, especially, for example, to detect novel viruses generated by recombination of human and animal viruses, such as human and avian viruses. Such methods can also be applied to an individual to select optimal therapeutic interventions and avoid the generation of resistant strains. The method can further include sequencing or other methods of analysis to confirm the identity of the viral sequences present.

The invention relates to the detection of viral pathogens not associated with cancer or other malignancies, including both known and unknown pathogens. For example, cervical cancer is related to infection with Human Papilloma Virus (HPV). Tumor biopsies and samples can be analyzed for the presence of viral pathogens, and the screening of a large number of samples can be performed to correlate the presence of a specific viral pathogen with the presence of a particular malignancy. The method includes isolation of nucleic acids from tissue or bodily fluid from individuals know to or suspected of having cancer; labeling the nucleic acids; contacting the labeled nucleic acids with a microarray of the invention; detecting the presence of a labeled nucleic acid hybridized to the array per the methods of the invention; and correlating the presence of a specific pathogen with the cancer. The method can further include sequencing or other methods of analysis to confirm the identity of the viral sequences present.

The invention further relates to methods for identification of sequences for inclusion in the microarrays of the invention. Microarrays including a plurality of n-mer viral nucleotides of conserved and non-conserved nucleotide regions of all known viruses as of May 2, 2006 can be generated by downloading viral genome sequences from NCBI; identifying overlapping probes with the length of n-mer basepairs and a moving window of 10 basepairs; and performing a BLAST search of all probes against each other, wherein the most non-conserved 5 pairs of probes are selected (+ strand and − strand). This process can be accomplished by manually selected sequences that cover specific regions that encode genes in the virus genome.

Alternatively, the nucleotide sequences of the plurality of n-mer viral nucleotides of both conserved and non-conserved nucleotide regions of known viruses are determined by steps comprising manually selected sequences which cover specific regions that encode genes in the virus genome.

The invention further relates to a method of designing n-mers for inclusion in a viral microarray, wherein the nucleotide sequences of the plurality of n-mer viral nucleotides are identical or complementary to conserved and non-conserved nucleotide regions of all known viruses as of May 2, 2006 are determined by steps comprising: downloading viral genome sequences from NCBI; identifying overlapping probes with the length of n-mer basepairs and a moving window of 8 to 12 basepairs; and performing a BLAST all probes against each other, wherein each the most conserved and non-conserved pairs of probes are selected (+ strand and − strand) for the microarray.

### BRIEF DESCRIPTION OF THE FIGURES

FIG. 1 is a schematic drawing of the viral microarray workflow, involving nucleic acid extraction, Cy3 labeling, hybridization, washing, detection, and database analysis.

FIG. 2 is an illustration of the virus microarray performance wherein cross-hybridization derived from other immunodeficiency virus is visible, reflecting the successful representation on the array of conserved regions within the human immunodeficiency/simian immunodeficiency (HIV/SIV) RNA virus family.

FIG. 3 is an illustration of hybridization results from two HeLa cell lines which harbors human papilloma virus (HPV) 18, a DNA virus, which shows detection of HPV 18 from both the cell lines and also detection of some other viruses in the lab strain; these results confirm the sensitivity of methodology in detecting possible unknown viruses.

FIG. 4 is an illustration of hybridization results from JSC-1 cell line that harbors Epstein Barr Virus (EBV) and Kaposi's sarcoma-associated herpesvirus (KSHV), both DNA viruses, and validates the use of the array to detect multiple viral co-infection in cultured cells.

FIG. 5 is an illustration of hybridization results from BCBL-1 cell line that harbors Kaposi's sarcoma-associated herpesvirus (KSHV).

### DETAILED DESCRIPTION OF THE INVENTION

The rapid development of genomic databases, bioinformatics tools, and enabling technologies such as cDNA and oligonucleotide microarrays have provided new insights and understanding into biological and disease processes thru the global analysis of nucleotide sequences. The present invention relates to DNA microarrays for viral detection. The viral microarray consists of all known mammalian and avian pathogenic viruses, approximately 600, represented by 10,000 DNA oligonucleotide features (see Tables 1-3). The oligonucleotide features are 50-80-mer long DNA sequences distributed across both conserved and non-conserved regions of known viruses. This design feature provides a validation mechanism via redundant signals associated with each virus represented and also facilitates the discovery of “new” viruses that have arisen via recombination. Viral sequences were obtained from GenBank.

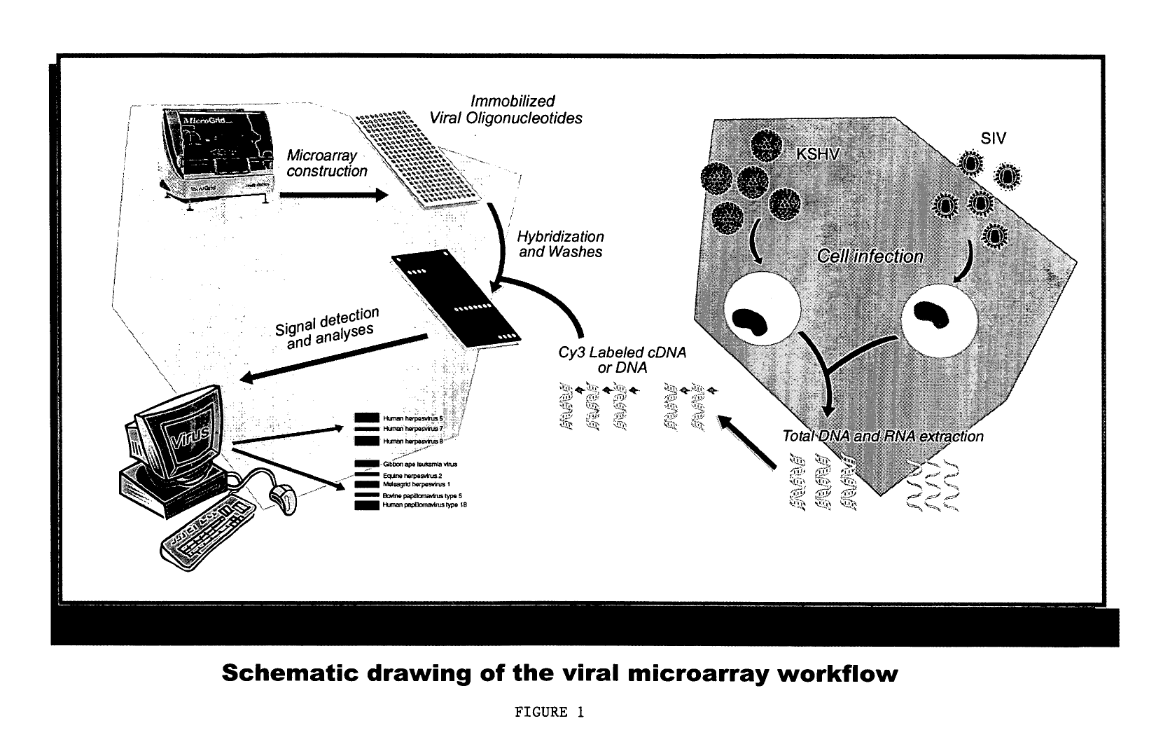

Positive and negative controls features are designed against human and mouse house-keeping genes such as actin, GAPDH, Line-Sine sequences, human endogenous retrovirus. The inclusion of human or other mammalian sequences in the microarrays of the invention is within the scope of the invention. Virus microarray detection performance was tested and validated through analysis of reverse transcribed RNA (i.e., cDNA) and DNA from tissue culture cells infected with different viruses. The schematic drawing of the viral microarray technology operation can be visualized in FIG. 1. Briefly, DNA or RNA is isolated from a sample(s). DNA is labeled with fluorescent dye (e.g., Cy3), and hybridized to the microarray. After washing, the microarray is scanned using an Agilent scanner to detect the bound, labeled nucleic acids from the sample. The positions of the fluorescent signals are correlated with specific sequences to which the labeled nucleic acids are hybridized. Results are analyzed using feature analysis program software. The labeled nucleic acids can be physically removed from the support and further analyzed by PCR and/or sequencing to confirm the sequence of the nucleic acid, or to identify new viral species or strains, or mutations within known viral species or strains.

The development of protocols for utilization of total DNA and RNA from samples for virus detection allow us to use the DNA microarray to detect the two different known classes of viruses: viruses of RNA and viruses of DNA. For the virus array of the invention, as little as 50 ng input of either total DNA or RNA extracted from virus infected cultured cells or other biological samples (e.g., samples obtained from subjects, cell or viral stocks) are necessary, representing as few as 5 to 10 viral particles or the virus to be detected. This technology, enables high-throughput screening that allows detection and identification hundreds of viruses. It can be used for detection and identification of viruses in diseases where no etiologic agent has been detected, for large-scale epidemiological studies, or for any of a number of other purposes such as those discussed herein. The arrays and methods of the invention are ideally suited for the detection of viral recombination due to the breadth of the viral types included in the array and the inclusion of both conserved and non-conserved sequences to allow for more definitive identification of hybridized viral sequences as compared to detection methods that include only conserved sequences. Moreover, the methods of the invention for the detection of DNA viruses do not rely upon amplification using sequence specific primers (e.g., PCR), significantly reducing the chance of introducing bias into the labeled nucleic acid sample.

With the viral arrays technology described herein, a diverse range of clinical and research samples can be screened in a high-throughput manner and a large number of samples can be analyzed in parallel on identical arrays. This technology can be very useful for biomedical research and clinical diagnostics. Since this DNA microarray can also be used for viral discovery in mouse and monkeys, it can be a diagnostic tool for the identification of pathological agents responsible for disease outbreaks in animal facilities. The ability to detect multiple, unrelated viruses in a single array is also an asset in such diagnostic methods when there may be little indication regarding the identity of the pathogen. Microarrays and methods of the invention also can be a used for the identification of viral agents that are a result of bioterrorism attacks wherein the potential pathogens can be a combination of agents or wherein there may be little or no suggestion regarding the type of pathogen released.

The viral microarray methods include a viral nucleic acid extraction step, a nucleic acid labeling step, a hybridization step, and a detection step. The methods can further include a sequencing step, and a sequence comparison step using known viral sequence databases to allow for confirmation of the identity of a hybridized sample, or the identification of new viruses.

The viral nucleic acid extraction from samples can be carried out by a number of methods currently known to one of ordinary skill in the art and optionally using kits that are commercially available. Once total viral nucleic acid has been extracted, all nucleotides from a particular sample are optionally amplified and labeled nucleic acids are prepared with a fluorescent dye, such as Cy3.

In the hybridization step, the test sample containing viral nucleotides is contacted with a viral microarray. If a nucleic acid is contained in the test sample hybridizes with (i.e., is sufficiently complementary to) at least one of the plurality of n-mer viral nucleotides immobilized on the viral microarray, it is bound to the microarray via that immobilized nucleic acid. In this case, hybridization between the viral microarray and sample viral nucleic acid is detected in the subsequent detection step.

In the detection step, a nucleotide sequence that is hybridized to the viral microarray is detected. This detection uses known detection means that can be applied to a microarray method, particularly fluorescence spectroscopy. The use of n-mers directed to both conserved and non-conserved viral sequences substantially reduces the need for sequencing or the use of other methods to specifically identify the virus present. The location of the labeled hybridized sequences on the microarray are used to identify the virus present. Following detection and identification, to confirm the identity of the hybridized, labeled nucleic acid, the detected sample viral nucleic acid can be sequenced and the sequence is compared to viral database sequences.

The microarrays and methods of the invention can be used for detection of viral sequences, including pathogenic viral sequences, in a number of samples from various sources.

The term “detection marker”, “detection label”, “detectable label” or other like term as used herein is understood as a tag such as a fluorescent, colormetric, enzymatic, or radioactive tag that can be readily observed by direct or indirect methods such as microscopy and/or exposure to film or other recording device such as a scanner. In a preferred embodiment of the invention, fluorescent tags are used. Fluorescent tags include, but are not limited to, Cy3, Cy5, Cy5.5, fluorescence, rhodamine, SYBR green, Texas Red, DyLight Reactive Dyes and Conjugates including DyLight 488, 549, 649, 680 and 800 Reactive Dyes, Alexa Dyes (Alexa 488, Alexa 546, Alexa 555, Alexa 647, Alexa 680) and IRDye 800. Nucleic acids are preferably labeled with detectable labels using modified nucleotide analogs including detectable labels. Alternatively, nucleic acids can be labeled using nucleotide analogs including groups that are the first half of a binding pair, such as biotin, to be reacted with a detectable label attached to the other half of the binding pair, such as strepavidin. Such nucleotide analog reagents are commercially available from a number of sources. “Labeled nucleic acids” are nucleic acids labeled with a detectable label. It is understood that labeling of a nucleic acid of the invention can include incorporation of a label or other modified nucleotide into a new nucleic acid molecule generated by a polymerase using the nucleic acid isolated from the sample as a template.

The term “detection”, “detect,” or variations thereof as used herein is understood to mean looking for a specific indicator of the presence of one or more nucleic acids bound to a specific location on the solid support corresponding to a specific n-mer. The amount of nucleic acid detected can be none, i.e., below the detection limit. The detection limit can depend on a number of factors including the efficiency and specific activity of the label, or tag used. The term “identification,” “identify,” or variations thereof is understood as the correlation of a specific location on the solid support to a specific nucleic acid. A nucleic acid sequence, which corresponds to at least one virus, is identified by correlating the presence of the detectable marker with the predetermined position of the corresponding n-mer on the support. As the specificity of hybridization can be varied, the relative binding to one position on the microarray to another can be determined. The identity of a labeled nucleic acid can be confirmed by removing the nucleic acid from the microarray and subjecting it to other methods such as sequencing or PCR.

The term “nucleic acid sample”, “sample nucleic acid”, or the like as used herein, may include any polymer, including pyrimidine and purine bases, preferably cytosine, thymine, and uracil, and adenine and guanine, respectively. See Lehninger, Principles Of Biochemistry, at 793-800 (Worth Pub. 1982). The sample nucleic acid is preferably a naturally occurring nucleic acid or fragment thereof, or a nucleic acid generated by a biosynthetic method using a naturally occurring nucleic acid or fragment thereof as a template. As used herein, a naturally occurring nucleic acid is understood as a nucleic acid isolated from a biological sample, such as a tissue or bodily fluid of a subject. For example, sample nucleic acids include DNA or RNA isolated from a biological sample, cDNA reverse transcribed from an RNA, a nucleic acid polymerization product generated using non-thermostable polymerases (e.g., Klenow, to generate labeled nucleic acids), or a thermostable polymerase (e.g., Taq, to amplify the amount of sample present). Such biosynthetic methods are well known to those skilled in the art and can be used alone or in combination with each other in the methods of the invention. Fragments can be generated by enzymatic methods (e.g., endonucleases), or amplification of less than full-length copies of nucleic acids by polymerases; and mechanical methods (e.g., shearing or sonication). Fragments can also be generated during the process of sample collection and preparation, and during isolation of sample nucleic acids.

“Oligonucleotide”, “n-mer oligonucleotide” and the like refer to a polymeric nucleotides of any length, either ribonucleotides, deoxyribonucleotides or peptide nucleic acids (PNAs), or a combination thereof, that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and are preferably artificially or synthetically produced. The oligonucleotides used in the present invention can be individually prepared by one of ordinary skill in the art, or they may be purchased, since many are commercially-available or can be ordered from companies that perform custom oligonucleotide synthesis.

The term “n-mer” as used herein, refers to an oligomer or polymer that is comprised of a series of monomers, preferably nucleotide monomers. The n-mers of the invention are preferably about 60 to about 70 nucleotides in length; however, other lengths are possible. For example, n-mers can be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length.

The term “nucleic acid microarray” or “viral microarray” as used herein, refers to an intentionally created collection of n-mer oligonucleotides that can be prepared either synthetically or biosynthetically and can be used to test for hybridization of nucleic acids from samples suspected of containing viral nucleic acids. Such arrays can also be screened for hybridization to a labeled nucleic acid sample in a variety of different formats (for example, libraries of soluble molecules; and libraries of oligos tethered to resin beads, silica chips, or other surfaces). Additionally, the term “array” is meant to include those libraries of nucleic acids that can be prepared by spotting nucleic acids of essentially any length (for example, from 1 to about 1000 nucleotide monomers in length) onto a substrate. In a preferred embodiment, the nucleic acids are arrayed in defined positions on a surface or support such that the identity of the nucleic acid can be determined by its position on the surface.

The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “surface,” “solid support,” “support,” and “substrate” as used herein, are used interchangeably and refer to a material or group of materials having a rigid or semi-rigid surface or surfaces such as nitrocellulose, nylon, polyvinylidene difluoride, glass, or plastics, and their derivatives. In the exemplified embodiment the substrate is a glass slide. In many embodiments, at least one surface of the solid support will be substantially flat, although in some embodiments it may be desirable to physically separate synthesis regions for different compounds with, for example, wells, raised regions, pins, etched trenches, or the like. According to other embodiments, support(s) will take the form of beads, resins, gels, microspheres, or other geometric configurations. See, e.g., U.S. Pat. No. 5,744,305 for other exemplary substrates. Technology was developed for making high density DNA microarray (Shalon et al., Genome Research, 1996 July; 6(7): 639645.). The present invention can also employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference. The method of the synthesis of the nucleotides of the array is not a limitation of the invention.

The term “sample” such as a sample from a subject, as used herein includes a tissue or bodily fluid of a subject, such as an animal, mammal, or preferably a human subject. The sample can be obtained from cultured cells, including primary or immortalized cell lines. A sample can include a biopsy or tissue removed during surgical or other procedures. Samples can include frozen samples collected for other purposes. Samples are preferably associated with relevant information such as age, gender, and clinical symptoms present in the subject; source of the sample; and methods of collection and storage of the sample,

The term “bodily fluid” is understood herein to mean any essentially liquid sample obtained from a subject, such as an animal, mammal, or preferably human subject, that may or may not contain cells. If the bodily fluid includes cells, the cells are preferably removed (e.g., by centrifugation or filtration) or extracted prior to contacting the bodily fluid with the microarray. Bodily fluids can include, for example, blood, serum, breast milk, semen, urine, sputum, vomit, and lymph. Bodily fluids are preferably diluted in an appropriate buffer before labeling or contacting the fluid with a microarray.

The term “isolated nucleic acid” as used herein, mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods). The term “mixed population” or “complex population” as used herein, refers to any sample containing both desired and undesired nucleic acids. As a non-limiting example, a complex population of nucleic acids may be total genomic DNA, total genomic RNA or a combination thereof. A complex population can also include both viral and host nucleic acids. Moreover, a complex population of nucleic acids may have been enriched for a given population, but also include other undesirable populations. For example, a complex population of nucleic acids may be a sample which has been enriched for desired messenger RNA (mRNA) sequences, but still includes some undesired ribosomal RNA sequences (rRNA). The oligonucleotide spots are preferably isolated nucleic acids.

The term “conserved sequences” or “conserved nucleic acid sequences” refers to nucleic acid sequences that are similar or identical sequences within multiple species or strains of organism, or within different nucleic acid molecules in the same organism. Cross species conservation of nucleic acid sequences typically indicates that a particular sequence may have been maintained by evolution despite speciation. The further back up the phylogenetic tree a particular conserved sequence may occur the more highly conserved it is said to be. Therefore, binding to a conserved nucleic acid sequence typically provides more general information about a sample than binding to a non-conserved sequence. The term “non-conserved sequences” or “non-conserved nucleic acid sequences” refers to nucleic acid sequences that are distinct between multiple species within a genus, and preferably between various viral strains within a species. The degree of conservation of nucleic acid sequences can be determined using any of a number of programs and methods including the BLAST sequence database available through the National Center of Biotechnology Information (NCBI) and ClustalW available through the European Molecular Biology Laboratory-European Bioinformatics Institute (EMBL-EBI). Other alignment tools and methods are known to those in the art.

The term “conditions to allow binding” or “conditions to allow hybridization” is understood herein as buffer, salt, detergent, and temperature conditions that permit specific hybridization of the n-mers with the labeled nucleic acids. Such conditions are well known to those skilled in the art and are discussed, for example in Molecular Cloning: A Laboratory Manual (Maniatis, Cold Spring Harbor Laboratory Press). It is understood that various conditions (i.e., stringencies) of hybridization and washing can be used to modulate the level of complementarity required for the hybridization of the n-mer to the labeled nucleic acid. A single microarray can be washed using progressively more stringent conditions to increase the degree of complementarity between the n-mer and the labeled nucleic acid. Preferred conditions for binding are discussed in the Examples below.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook et al., Molecular Cloning A laboratory Manual, 2nd Ed., Cold Spring Harbor Press (1989), herein incorporated by reference in its entirety.

The term “hybridization conditions” as used herein will typically include salt concentrations of less than about 1M, usually less than about 500 mM, and preferably less than about 200 mM. When the term “effective amount” is used herein, it refers to an amount sufficient to induce a desired result. Hybridization temperatures can be as low as 5° C., but are typically >22° C., more typically >30° C., and preferably >37° C. Longer sequence fragments may require higher hybridization temperatures for specific hybridization. Other factors may affect the stringency of hybridization, including base composition and length of the complementary strands, presence of organic solvents and extent of base mismatching, as a result the combination of parameters is more important than the absolute measure of any one alone. Such considerations are well known and understood by those skilled in the art. Preferred conditions for hybridization are provided in the examples below.

The term “hybridization” as used herein, refers to the process in which two single-stranded polynucleotides bind non-covalently to form a stable double-stranded polynucleotide. Triple-stranded hybridization is also theoretically possible, but it not preferred in the methods of the instant invention. The resulting (usually) double-stranded polynucleotide is a “hybrid.” The proportion of the population of polynucleotides that forms stable hybrids is referred to herein as the “degree of hybridization.”

The term “hybridization probe” as used herein, refers to an oligonucleotide capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. An n-mer of the invention can act as a hybridization probe. The term “hybridizing specifically to” as used herein, refers to the binding, duplexing, or hybridizing of a molecule only to a particular nucleotide sequence or sequences under stringent conditions when that sequence is present in a complex mixture (for example, total cellular) DNA or RNA. It is understood that sequences do not need to be 100% complementary to specifically hybridize.

The term “overlapping probe” as used herein is understood as a series of probes designed by performing an 8, 9, 10, 11 or 12 basepair “walk,” preferably a 10-basepair “walk” along a viral sequence. The length of the overlap depends on the length of the n-mers to be designed. The amount of overlap equals the length of the n-mer minus the length of the “step” in the “walk.” Typically overlap is about 40 to about 70 basepairs.

The term “plus strand” or “+strand” as used herein is understood to be the coding or sense strand of the viral sequences. “Minus strand” or “− strand” as used herein is understood to be antisense or non-coding strand of the viral sequence.

The term “target” as used herein refers to a molecule that has an affinity for a given probe. For example, a nucleic acid hybridizes, preferably specifically hybridizes, to its target nucleic acid. Targets may be naturally-occurring or man-made molecules. Also, they can be employed in their unaltered state or as aggregates with other species. Targets may be attached, covalently or noncovalently, to a binding member, either directly or via a specific binding substance. Examples of targets that can be employed in the instant invention are nucleic acid molecules including natural and non-natural nucleotides and nucleotide analogs prepared by recombinant or synthetic methods. Nucleotide analogs include nucleotides that can be incorporated into nucleic acid molecules and base pair with a complementary strand. Non-natural nucleotides and nucleotide analogs can include sugar, base, and/or backbone modifications relative to natural nucleotides. Targets are sometimes referred to in the art as anti-probes. As the term targets is used herein, no difference in meaning is intended. A “probe to-target pair” is formed when two macromolecules have combined (e.g., hybridized) through molecular recognition to form a complex.

The term “complementary” as used herein, refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of tbe nucleotides of the other strand, usually at least about 90% to 95%, and more preferably from about 98% to 100%. Percent complementarity can be readily determined by dividing the number of complementary nucleotide pairs over the length of the shorter nucleic acid by the overall length of the shorter nucleic acid. Percent complementarity can also be determined using computer programs such as BLAST available through the NCBI. Methods of determining percent complementarity are well known and understood by those skilled in the art.

Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, e.g., Kanehisa, Nucleic Acids Res. 12:203 (1984), incorporated herein by reference.

The term “monomer” as used herein, refers to any member of the set of molecules that can be joined together to form an oligomer or polymer. The set of monomers useful in the present invention includes, but is not restricted to, for example, nucleic acid polymer synthesis, the set of natural and modified nucleic acids; and (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. As used herein, non-natural nucleotides include nucleotides that have sugar, backbone, or base modifications to alter at least one property of the nucleotide including, but not limited to, stability, affinity for a target or complementary sequence, and/or to provide a new function to the nucleotide polymer such as strepavidin binding by including monomers having a biotin group. Different basic sets of monomers may be used at successive steps in the synthesis of a polymer. The term “monomer” also refers to a chemical subunit that can be combined with a different chemical subunit to form a compound larger than either subunit alone.

The term “viral nucleotides” include sequences identical or complementary to viral sequences, for example from the sequences defined by the GenBank numbers included in Table 1 and the sequences in Table 2.

The term “viruses associated with a particular disease or disorder” refers to the correlation or the presence of certain viral pathogens with certain signs, symptoms, or indicators of specific diseases. These correlations are well known. For example, Human and Simian Immunodeficiency Viruses (HIV and SIV, respectively) are known to be associated with acquired immunodeficiency disease. Hepatitis viruses are known to be associated with hepatic (liver) disease. SARS and Respiratory Syncytial Virus (RSV) are known to be associated with pulmonary disease. Rhinovirus is associated with the common cold.

The term “obtaining” as in “obtaining a nucleic acid” or “obtaining a sample” refers to purchasing, synthesizing, removing from a subject, or otherwise procuring an agent, sample, or nucleic acid.

The term “subject” refers to an animal, preferably a mammal including a human. A subject is a source for cells, bodily fluids, and/or tissues for the preparation of isolated nucleic acids for use in the methods of the invention. A subject can also be an individual suspected of having a predisposition to a disease or disorder; or suspected of having a disease or disorder, such as a viral infection. A subject can be an individual having a predisposition to a disease or disorder, or having a disease or disorder. Human subjects suspected of or known to have a disease, disorder, or infection can be referred to as “patients.” An individual having a predisposition to a disease or disorder or suspected of having a disease or disorder can be identified using, for example, standard diagnostic methods.

The term “diagnosis”, “diagnosing”, and the like are understood to mean to recognize (as a disease) by signs and symptoms a disease or condition in a subject or patient, or to analyze the cause or nature of a problem, particularly a physiological problem. Diagnosis does not require a conclusive indication of disease. Diagnosis can be a process. Identification of one or more pathological viral sequences in a sample from a subject can be used for or contribute to the diagnosis of a disease.

The term “normal tissue” or “non-cancerous tissue” and the like are understood herein to mean tissue that has no apparent growth abnormalities or other signs of carcinoma, neoplasia, or dysplasia (e.g., loss of ploidy, dedifferentiation). Normal tissue can be derived from a disease free individual, or from an individual having disease from a location not showing signs of disease. The term “non-normal tissue” or “non-cancerous tissue” and the like are understood to mean tissue that does not show growth abnormalities or other signs of carcinoma, neoplasia, or dysplasia.

The term “plurality” is understood to mean more than one.

The terms “a” and “the” are understood to be both single and plural unless otherwise indicated by context. The term “or” is understood to be inclusive unless otherwise indicated by context.

Ranges are understood to include all of the numbers within the range. For example, 1 to 50 is understood to include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 41, 42, 43, 44, 45, 46, 47, 48, 49, and 50.

The practice of the present invention may also employ conventional biology methods, software, and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example, Setubal et al., Introduction to Computational Biology Methods (PWS Publishing Company, Boston, 1997); Salzberg, Searles, Kasif, (Ed.); Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis, Bioinformatics: A Practical Guide for Analysis of Gene and Proteins (Wiley & Sons, Inc., 2.sup.nd ed., 2001). See also, e.g., U.S. Pat. No. 6,420,108.

The present invention may also make use of various computer program products and software for a variety of purposes, such as probe design, management of data, analysis, and instrument operation. See, e.g., U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559, 10/065,856, 10/065,868, 10/328,818, 10/328,872, 10/423,403, and 60/482,389.

Additional objects, advantages and novel features of the invention will become apparent to those skilled in the art on examination of that described herein, or may be learned by practice of the invention.

### EXAMPLE 1

### Preparation of Labeled DNA Samples from Genomic DNA for Use with Microarrays

Whole genomic DNA is isolated from a sample using any of a number of well-known methods or commercially available kits. The exact method of DNA isolation is not a limitation of the invention. For example, whole genomic DNA was isolated from virally infected cells using the QIAamp DNA Minikit (Qiagen, Hilden, Germany).

Labeled nucleic acids for hybridization were generated from the isolated DNA templates by incorporation of fluorophor-labeled dCTP in a random primed polymerization reaction using a Bioprime DNA labeling kit (Invitrogen, Carlsbad, Calif.). All steps were performed to minimize exposure to light after addition of the fluorophor labeled nucleotide. Briefly, 0.1-1 μg of genomic DNA digested with EcoRI was incubated with 60 μM of either Cy3- or Cy5-dCTP (Perkin-Elmer); 120 μM each dATP, dGTP, dTTP and 60 μM dCTP; 30 μl of 2.5×random nonamers (9 nucleotide random sequence oligonucleotides) (Invitrogen), and 40 units of Klenow DNA polymerase in a 50 μl total reaction volume. The mixture was incubated at 37° C. for about 16 hours.

The reaction was stopped by heating the mixture to 65° C. for 20 minutes. The random hexamers and unincorporated nucleotides were removed by gel filtration with a Bio-Gel P6 column (BioRad, Hercules, Calif.) per manufacturer's instructions. Samples were dried by vacuum and dissolved in 75 μl H₂O.

### EXAMPLE 2

### Preparation of Labeled DNA Samples from RNA for Use with Microarrays

RNA can be isolated from a sample using any of a number of well-known methods or commercially available kits. The exact method of RNA isolation is not a limitation of the invention.

For example, RNA was isolated from FluMist® (Influenza Virus Vaccine Live, Intranasal; MedImmune Vaccines, Inc) and from human samples including saliva, swabs, and vomit, using a modified version of the viral extraction protocol of Lai and Chambers (Biotechniques, 19:704-706, 1995). Briefly, 50 mM Tris, pH 7.4 was added to each 500 μl sample. The sample was incubated at 37° C. for 3 hours, and phenol-chloroform extracted. The aqueous layer was precipitated using sodium acetate, pH 5.3, and absolute ethanol. The samples were centrifuged at 14,000×g for 15 minutes at 4° C. to pellet the nucleic acids. The pellet was washed with 70% ethanol, dried, and resuspended in water. After RNA extraction, RNA was subject to reverse transcription and polymerase chain reaction. Briefly, RNA was reverse transcribed using 40 pmol/μl of a primer 5′-GTT TCC CAG TCA CGA TAN NNN NNN. Second strand synthesis was carried out with 8 units of Sequenase (United States Biochemical). Subsequently, the 30 μl reaction mixture was used as a template for PCR amplification (40 cycles, 30 s at 94° C., 30 s at 40° C., 30 s at 50° C., and 60 s at 72° C.) with 100 pmol/μl using the following primer 5′-GTT TCC CAG TCA CGA TC.

A second series of amplification cycles was performed including 20 additional PCR cycles as described above to incorporate amoniallyl-dUTP (Sigma). The aminoallyl containing cDNAs were purified using CyScribe GFX Purification Kit (GE-Amersham) per manufacturer's instructions. Purified products were labeled with n-hydroxyl succinimide (NHS) ester of Cy3 or Cy5 following manufacturer's instructions. Unincorporated nucleotides and fluorophors were removed using CyScribe GFX Purification kit (GE-Amersham). Samples were dried and resuspended in water.

Labeled nucleic acid yield was quantified by spectrophotometric absorbance at wavelengths 550 nm and 650 nm to quantitate the amount of Cy3 or Cy5 present in the sample, respectively.

### EXAMPLE 3

### Preparation of Microarrays

The virus microarray is printed via a contract with Agilent Technologies (Palo Alto, Calif., USA). The 60-mer oligos were synthesized on glass slides using Agilent's non-contact in situ synthesis process of printing 60-mer length oligonucleotide, base-by-base, from digital sequence files. The virus microarray slides contain two arrays where up to 11,000 oligonucleotides per array can be synthesized (2X11K format).

### EXAMPLE 4

### Annealing, Hybridization, and Detection of Labeled Nucleic Acids on Microarrays

Hybridization, washing, and drying of the microarrays was performed essentially according to Agilent instructions with some modifications.

Briefly, 75 μl labeled DNA prepared from DNA or RNA templates was combined with 25 μl human Cot-1 DNA (Invitrogen; placental DNA 50-300 bp in length enriched for repetitive sequences for use as a blocking agent); 25 μl Agilent blocking agent; and 125 μl Agilent 2×hybridization buffer. The mixture was heated to 95° C. for 3 minutes to denature the DNA, and subsequently incubated at 37° C. for 2 hours to allow hybridization of repetitive sequences of the labeled nucleic acid to the Cot-1 DNA. The mixture was centrifuged at 14,000×g to remove any precipitates.

Hybridization was performed in an Agilent hybridization chamber for at least 16 hours in a 65° C. rotating oven at 10 rpm (SciGene, Sunnyvale, Calif.). After hybridization, slides were washed in 5×SSPE, 0.0005% N-laurylsarcosine (SDS); followed by 0.1×SSPE, 0.0005% N-laurylsarcosine (SDS). Washes are preformed at 65° C. An additional wash was performed at room temperature in Agilent stabilizer for 1 minute. Slides were dried and subject to fluorescent detection using an Agilent Microarray Scanner. The presence and concentration of the DNA virus was independently confirmed and analyzed by conventional PCR.

### EXAMPLE 5

### Hybridization of Viral Sequences to both Conserved and Non-Conserved Sequences

Total RNA was isolated from the Cem X174 cell line which harbors SIV to determine if the microarrays and methods of the invention could be used for the detection of nucleic acids from RNA viruses.

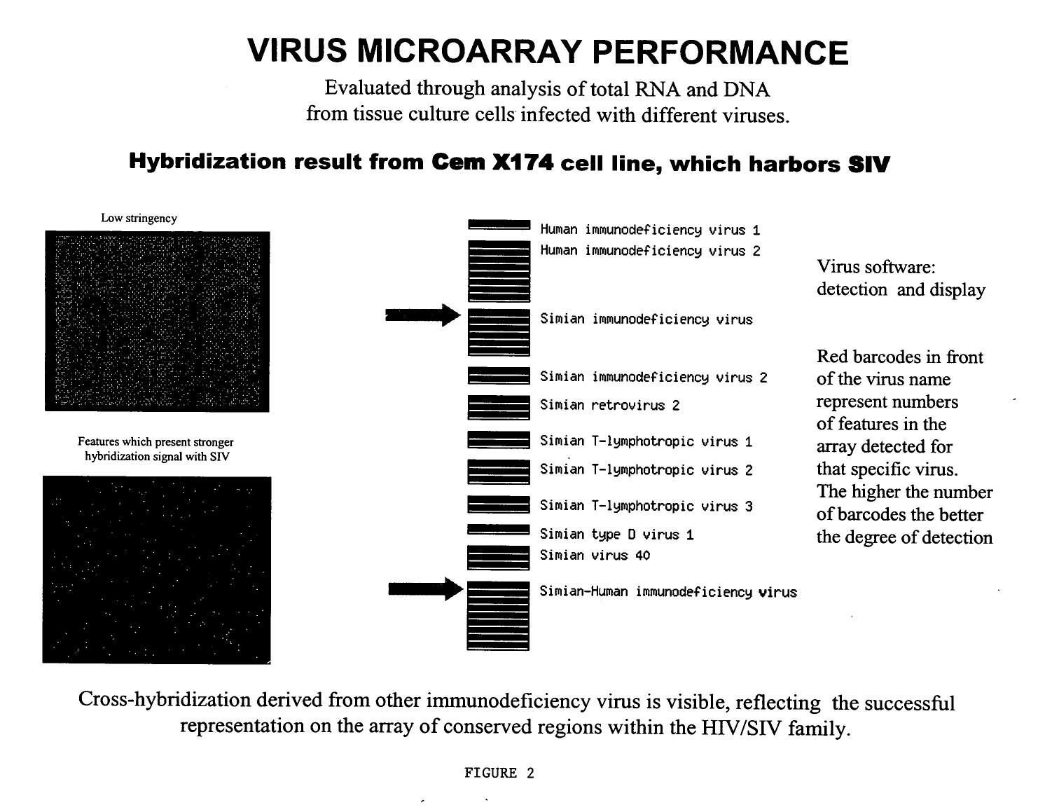

The RNA was reverse transcribed and amplified essentially as set forth in the examples above. The labeled nucleic acid was contacted with the microarray of the invention. Hybridization, washing, and detection steps were performed essentially as set forth above. The top panel subject to only a low stringency wash shows a large number of spots where the labeled nucleic acid is hybridized. The lower panel shows a separate experiment in which a full series of washes was performed. Specific hybridization of the labeled nucleic acids to the array is observed. Hybridized labeled nucleic acids were detected and identified by their position on the microarray. The spots were correlated with specific sequences as shown in FIG. 2. The number of bars indicates the number of features for a specific virus detected in the sample. As expected, some cross-hybridization was observed with sequences from similar viruses. However, the largest number of features bound by the labeled nucleic acid were those of SIV. This demonstrates that the inclusion of non-conserved sequences in the microarray improves specific identification of viruses as compared to microarrays and methods that include only conserved sequences.

### EXAMPLE 6

### Detection of Contaminating Viral Sequences in a HeLa Cell Laboratory Strain

Laboratory cell strains are passaged multiple times, often by multiple individuals, in common tissue culture hoods. This provides opportunities for contamination of cell lines with viruses, other cell lines, and other contaminants that may not be seen by standard cell monitoring (e.g., light microscopy).

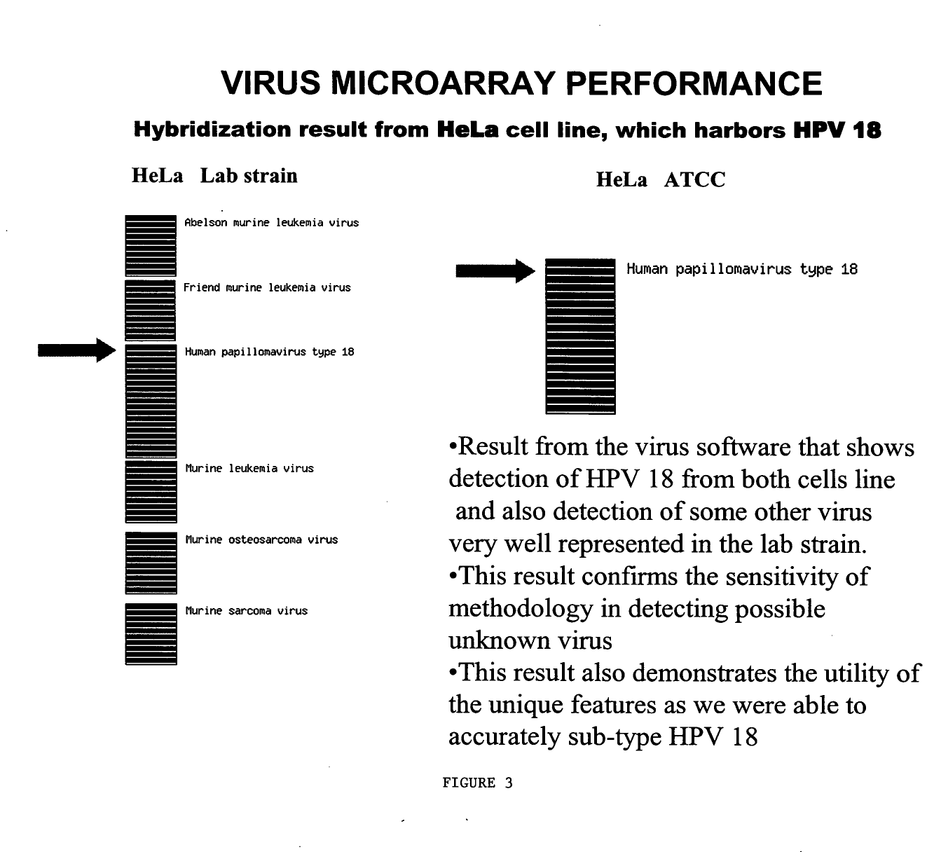

A laboratory strain of HeLa cells was tested for contamination against a freshly obtained aliquot of HeLa cells from a commercial source (ATCC). HeLa cells harbor HPV 18, but should not include any other viruses. Total genomic DNA was isolated from the two cell lines and labeled essentially as described above. The labeled nucleic acid were subject to analysis using the microarrays and methods of the invention. FIG. 3 is an illustration of hybridization results from the two HeLa cell lines shows detection of HPV 18 from both the cell lines. However, the laboratory strain was found to include contaminants having sequences that hybridize to sequences from at least another 5 viruses. These results confirm the sensitivity of methodology in detecting possible unknown viruses and the ability to detect multiple unrelated viruses and/or viral sequences in a single sample.

### EXAMPLE 7

### Detection of Viral Sequences in JSC-1 Cells that Harbor Two Viruses

The JSC-1 cell line harbors two viruses, Epstein Barr Virus (EBV), also known as human herpes virus 4; and Kaposi's sarcoma-associated herpes virus (KSHV), also known as human herpes virus 8. Both are DNA viruses.

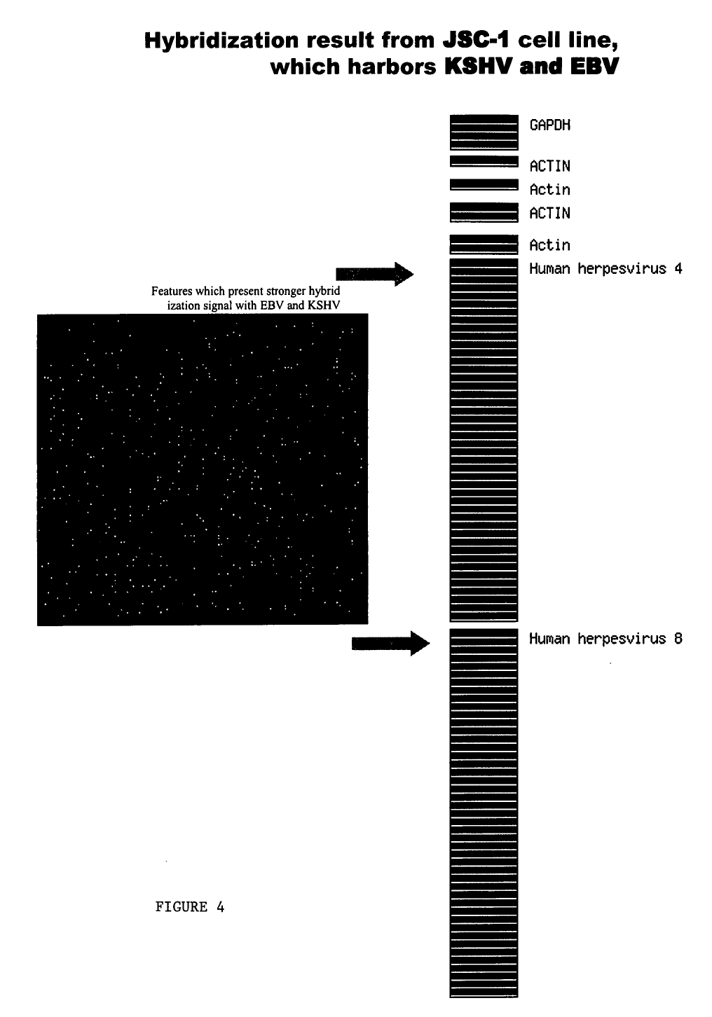

Total genomic DNA was isolated from the cells and labeled essentially as described above to determine if the arrays and methods of the invention could be used to identify viral sequences from cells carrying multiple viruses. The labeled nucleic acid was subject to analysis using the microarrays and methods of the invention. FIG. 4 is an illustration of hybridization results from JSC-1 cell line. The data validates the use of the array to detect and distinguish between multiple viruses of the same family in a single sample.

### EXAMPLE 8

### Detection of KSHV Sequences in BCBL-1 Cells

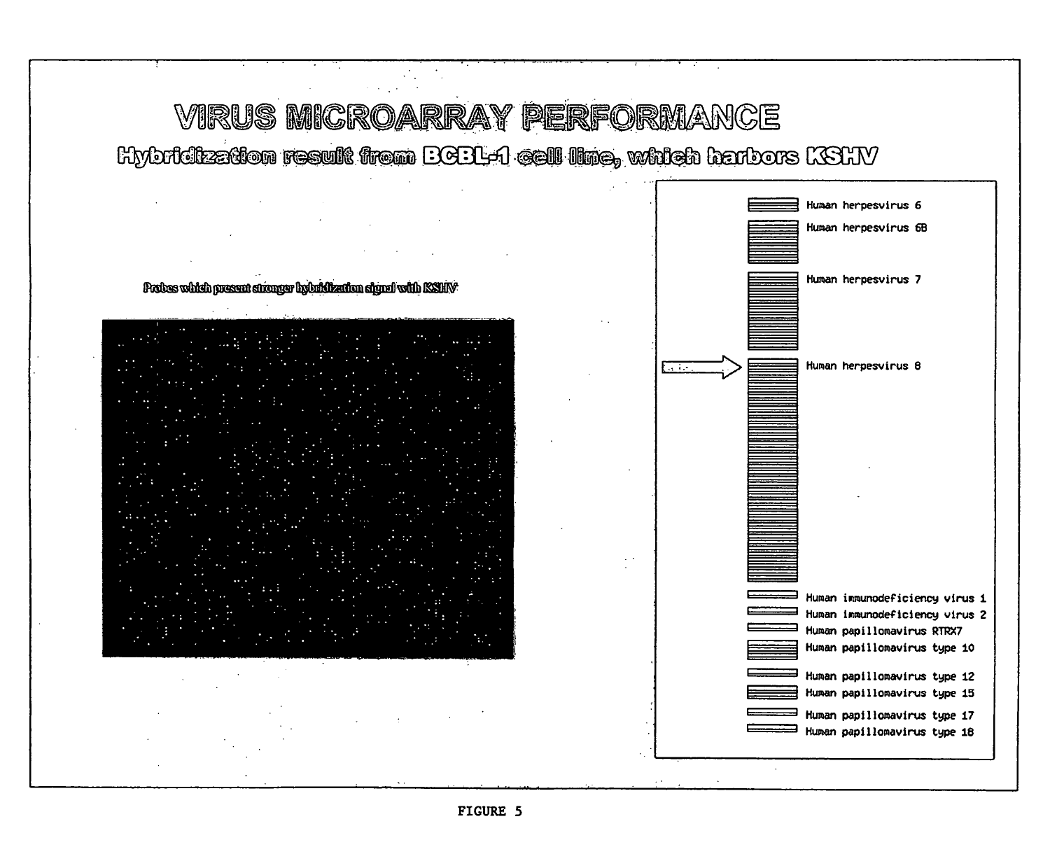

BCBL-1 cells harbor the KSHV virus. Total genomic DNA was isolated from the BCBL-1 cells and labeled essentially as described above. The labeled nucleic acid was subject to analysis using the microarrays and methods of the invention. FIG. 5 is an illustration of hybridization results from BCBL-1 cell line that harbors KSHV. This further demonstrates the effectiveness of the microarrays and methods of the invention for detection and identification of viruses.

### EXAMPLE 9

### Detection of Contaminating Viruses in Flumist®

FluMist® (Influenza Virus Vaccine Live, Intranasal; MedImmune Vaccines, Inc) is an intranasal vaccine for seasonal flu. The vaccine was tested for the presence of contaminating viral sequences and subsequently viruses.

Total genomic DNA was isolated from about 25% of a dose of FluMist®, labeled, and subjected to analysis using the microarray of the invention as set forth above. Labeled nucleic acids were found to hybridize to multiple influenza sequences of multiple types, as expected. However, labeled nucleic acids were also found to bind to seven human herpes virus 6 (HHV6) sequences and two human herpes virus 7 sequences.

Viral detection and identification was confirmed by “rescuing” the labeled nucleic acid from the microarray by physically collecting the sample with a scalpel. The labeled nucleic acid sample was amplified by PCR and subcloned into an appropriate vector for sequencing. A 240 basepair fragment was identified and found to have a sequence identical to HHV6. The amount of HHV6 sequence present in the sample was further quantitated by PCR using primers directed to various positions in the HHV6 genome. The presence of sequences to four different loci within the HHV6 genome was confirmed.

These data demonstrate that the microarrays and methods of the invention are capable of detecting small amounts of viral contaminants in samples containing a large excess of other viral sequences. The results further demonstrate the presence of contaminants in viral stocks and the importance of monitoring viral stocks, especially those used in the preparation of vaccines, for the presence of contaminating sequences.

### EXAMPLE 10

### Detection of Viral Sequences in Human Tissue for the Detection and Identification of Viruses Associated with Cancer

Some types of cancer are strongly associated with the presence of viruses. For example, cervical cancer is associated with the presence of Human Papilloma Virus (HPV). Human Papilloma Virus infection is considered a necessary but not sufficient cause of cervical cancer. Cervical Intraepithelial Neoplasias (CIN 1, CIN 2 and CIN 3) precede cervical cancer, which can often be cured with adequate treatment. The microarrays and methods of the invention were used to test for the presence of additional viral sequences in CIN tissue to determine if HPV or other viruses were present in the precancerous neoplasic tissue.

Tissue samples were collected from 41 patients with either CIN1 or CIN2 and 20 healthy (control) women. Blood sample and normal cervical tissue was collected from all participants and lesion biopsies were collected from the CIN patients. Total genomic DNA was isolated and labeled essentially as described above. The labeled nucleic acid was subject to analysis using the microarrays and methods of the invention. Samples were also subjected to analysis using an HPV hybrid capture 2 assay, an in vitro nucleis acid hybridization assay with signal amplification using microplate chemiluminescence for the quantitative detection of HPV in tissue specimens.

The data are presented below.

Virus Microarray

| | HPV+ | HPV− | Total |
|---|---|---|---|
| Patients | 31 (75%) | 10 | 41 |
| Controls | 4 (20%) | 16 | 20 |

Hybrid Capture 2 Results

| | HPV+ | HPV− | Total |
|---|---|---|---|
| Patients | 11 (27%) | 30 | 41 |
| Controls | 4 (20%) | 16 | 20 |

These data demonstrate that the microarrays and methods of the instant invention are more sensitive having a lower number of false negative results than the Hybrid Capture 2 which is know to have a relatively high rate of false negative results (see, e.g., Lonely et al., J. Low. Genit. Tract. Dis. 8:285-91, 2004). The data demonstrate the utility of the microarrays and methods of the invention for the diagnosis and prognosis of disease.

The microarrays were further analyzed for the presence of other viral sequences and their possible correlation with disease (p value).

| | % positive | % positive | Assoc. |
|---|---|---|---|
| Virus detected | (patients) | (controls) | w/disease |
| HPV | 75% | 20% | 0.01 |
| HHV-3 | 42% | 15% | 0.04 |
| H. Enterovirus A | 32% | 10% | 0.06 |
| JC polyomavirus | 52% | 25% | 0.05 |
| Hepatitis GB virus | 32% | 5% | 0.02 |
| HHV6 | 70% | 70% | — |
| HTLV-I | 45% | 45% | — |
| HTLV-2 | 59% | 65% | — |

These data demonstrate a correlation between the presence of four viruses in addition to HPV with neoplastic cervical tissue. The identification of such viruses can allow for new and better methods of detection and diagnosis of cervical cancer to allow for earlier detection. The identification of such viruses can also allow for the development of vaccines and other therapeutics for the treatment of cancer.

### EXAMPLE 11

### Detection and Identification of Viral Pathogens Associated with Cancers

The results of the previous example demonstrate that the methods and arrays of the invention can also be used for the detection of novel viruses associated with cancers not previously associated with viruses. For example, normal and abnormal (e.g., transitional lesions, neoplasia, carcinoma) tissues are isolated from subjects without and with disease. Total genomic DNA is isolated from normal and abnormal tissue, labeled, and subject to analysis using microarrays as set forth above.

Samples are analyzed for the presence of viral sequences in sufficient numbers to allow for statistically significant results to be obtained regarding the association of the presence of a virus with a specific cancer type. As shown above, viral pathogens can be present in normal samples that are not associate with cancer. The identification of such viruses can allow for new and better methods of detection and diagnosis of cancer to allow for earlier detection. The identification of such viruses can also allow for the development of vaccines and other therapeutics for the treatment of cancer.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

LENGTHY TABLE REFERENCED HERE
US20080003565A1-20080103-T00001
Please refer to the end of the specification for access instructions.

LENGTHY TABLE REFERENCED HERE
US20080003565A1-20080103-T00002
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20080003565A1). An electronic copy of the table will also be available from the USPTO upon request and payment of the fee set forth in 37 CFR 1.19(b)(3).

We claim:

1. A microarray comprising a surface with a plurality of n-mer viral nucleotides of conserved and non-conserved nucleotide regions of all known viruses as of May 2, 2006.

2. The microarray of claim 1, wherein the plurality of n-mer viral nucleotides are comprised of conserved and non-conserved nucleotide regions of viruses listed in Table 1.

3. The microarray of claim 1, comprising a plurality of n-mer viral nucleotides of conserved and non-conserved nucleotide regions of viruses in Table 1 associate with a particular disease or disorder.

4. The microarray of claim 1, wherein the n-mers of conserved and non-conserved nucleotide regions are selected from a list comprising n-mers in Table 2.

5. The microarray of claim 1, wherein the disease or disorder is acquired immunodeficiency disease.

6. The microarray of claim 1, wherein the n-mer viral nucleotides are comprised of about 50 to about 80 nucleotides in length.

7. The microarray of claim 1, wherein the n-mer viral nucleotides are comprised of about 60 to about 70 nucleotides in length.

8. The microarray of claim 1, wherein the nucleotide sequences of the plurality of n-mer viral nucleotides of conserved and non-conserved nucleotide regions of all known viruses are determined by steps comprising manually selected sequences which cover specific regions that encode genes in the virus genome.

9. The microarray of claim 1, wherein said surface is made of materials selected from the group consisting of nitrocellulose, nylon, polyvinylidene difluoride, glass, or plastics, and their derivatives.

10. The microarray of claim 1, wherein the number of said n-mer nucleotides immobilized on said surface ranges from 100 to 10,000 different kinds.

11. The microarray of claim 1, wherein the number of said nucleotides immobilized on said surface ranges from 500 to 5,000 different kinds.

12. A method for identifying known and unknown subtypes of mammalian and avian viruses comprising the steps of:
obtaining a microarray of claim 1;
isolating nucleic acids from a sample containing a virus and labeling the nucleic acid sequences with a detectable marker;
contacting the labeled nucleic acids from the sample to said surface with immobilized known conserved and non-conserved n-mer viral nucleotides and incubating under conditions to permit hybridization of said labeled nucleic acids thereto;
washing the surface, detecting hybridization of said labeled nucleic acids; and
identifying the nucleic acids based on their position bound to the array.

13. The method of claim 12 further comprising determining nucleic acid sequences of the detected hybridized nucleic acids and comparing the sequences with a database to identify the virus or new subtype virus.

14. The method of claim 12 further comprising confirming the identity of the detected nucleic acid using PCR.

15. A method of diagnosing a patient with a viral infection comprising the method of claim 12 wherein the virus is identified.

16. A method for identifying viruses associated with cancer comprising the steps of:
obtaining a plurality of microarrays of claim 1;
obtaining a plurality of normal samples comprising non-cancerous tissue and a plurality of non-normal samples comprising cancerous tissue;
isolating nucleic acids from the normal and non-normal samples and labeling the nucleic acid sequences with a detectable marker;
contacting the labeled nucleic acids from the samples to said surface with immobilized known conserved and non-conserved n-mer viral nucleotides and incubating under conditions to permit hybridization of said labeled nucleic acids thereto;
washing the surface, detecting hybridization of said labeled nucleic acids; and
identifying the nucleic acids based on their position bound to the array.

17. The method of claim 16 further comprising determining nucleic acid sequences of the detected hybridized nucleic acids and comparing the sequences with a database to identify the virus or new subtype virus.

18. The method of claim 16 wherein the cancer is cervical cancer.

19. A method for identifying contaminants in a biological sample comprising the steps of:
obtaining a microarray of claim 1;
isolating nucleic acids from a sample containing a virus and labeling the nucleic acid sequences with a detectable marker;
contacting the labeled nucleic acids from the sample to said surface with immobilized known conserved and non-conserved n-mer viral nucleotides and incubating under conditions to permit hybridization of said labeled nucleic acids thereto;
washing the surface, detecting hybridization of said labeled nucleic acids; and
identifying the nucleic acids based on their position bound to the array.

20. The method of claim 19, further comprising determining nucleic acid sequences of the detected hybridized nucleic acids and comparing the sequences with a database to identify the virus or new subtype virus.

21. The method of claim 19, wherein the biological sample is a viral stock.

22. The method of claim 19, wherein the biological sample is a cell line.

23. A method of designing n-mers for inclusion in a viral microarray, wherein the nucleotide sequences of the plurality of n-mer viral nucleotides are identical or complementary to conserved and non-conserved nucleotide regions of all known viruses as of May 2, 2006 are determined by steps comprising:
downloading viral genome sequences from NCBI;
identifying overlapping probes with the length of n-mer basepairs and a moving window of 8 to 12 basepairs; and
performing a BLAST all probes against each other, wherein each the most conserved and non-conserved pairs of probes are selected for the microarray.

24. The method of claim 23 when the window is about 10 basepairs.

25. The method of claim 23 wherein the number of pairs of probes selected is 4 to 6 pairs.
